(19) United States
(12) Patent Application Publication
(10) Pub. No.: US 2003/0097055 A1
(43) Pub. Date: May 22, 2003

(54) Method of reviewing tomographic scans with a large number of images

(75) Inventors: Jeffrey Harold Yanof, Solon, OH (US); David Morgan Kwartowitz, Elmont, NY (US); Shalabh Chandra, Twinsburg, OH (US)

Correspondence Address: Thomas E. Kocovsky, Jr., FAY, SHARPE, FAGAN, MINNICH & MckEE, LLP, Seventh Floor, 1100 Superior Avenue, Cleveland, OH, 44114-2518, US

(73) Assignee: Philips Medical Systems(Cleveland), Inc.

(21) Appl. No.: 09/990,518

(22) Filed: Nov. 21, 2001

(51) Int. Cl. A61B 5/05

(52) U.S. Cl. 600/407

(57) ABSTRACT

A diagnostic medical imaging system (10) includes an imaging apparatus (100) having an examination region (112) in which a subject (20) being examined is portioned. The imaging apparatus (100) obtains, at first resolution, a plurality of first image slices of the subject (20). The first image slices are loaded into a storage device, and a data processor combines subsets of first image slices to generate a plurality of second image slices having a second resolution lower than the first resolution. The subsets each includes a number n of contiguous first image slices. A display (152) having a plurality of view ports including a first view port which depicts one or more selected second image slices and a second view port which depicts one or more first image slices which are constituents of one of the second image slices depicted in the first view port.

### BACKGROUND OF THE INVENTION

The present invention relates to diagnostic medical imaging. It finds particular application in conjunction with computed tomography (CT) imaging and will be described with particular reference thereto. However, it is to be appreciated that the present invention is also amenable to other like applications using different imaging modalities, such as, x-ray imaging, continuous CT (CCT), magnetic resonance imaging (MRI), fluoroscopy, single photon emission CT (SPECT), positron emission tomography (PET), and the like.

Diagnostic medical imaging, and CT imaging in particular, is a valued tool for medical diagnosis, treatment planning and the like. The advent of what is known as multi-slice or multi-detector CT enables an increased anatomical coverage and/or increased longitudinal (z-axis) resolution relative to so called single-slice CT. However, the data acquisition in multi-slice CT typically results in hundreds of thin (e.g., on the order of 0.5 mm thick) axial cross-section slices/images per case or scan. At the rate CT scanner technology is developing, it is foreseeable that CT scanners will be capable of rapidly acquiring resolutionally isotropic scans, e.g., of the entire abdomen and thorax in less than a few minutes or even seconds. Even assuming a thickness of 0.5 mm, over a 20 cm range, 400 axial cross-section slices/images would be generated.

The higher z-axis resolution and/or the larger area coverage are generally welcome improvements. However, the vast number of images can at times be overwhelming and/or unduly burdensome. For example, it can be overly time consuming to review a large numbers of images when the high resolution is not always desired for every portion of the anatomy scanned. However, with the reduced resolution of thicker slices, small (i.e., having a thickness less than the slice thickness) tumors or other small anatomical features may become lost or imperceivable.

There exists in the prior art a trade-off between the level of resolution desired and the number of images which are generate. That is to say, high resolution is conventionally achieved at the cost of having to review larger numbers of images, and conversely, reviewing a smaller number of images is conventionally achieved at the cost of lower resolution. Accordingly, it is advantageous to a radiologist or other like professional to review image intensive cases with sufficient diagnostic accuracy and speed and, furthermore, to utilize a higher z-axis resolution during image review when it is desired without getting overwhelmed with too many images when higher resolution is not desired. It is further advantageous, to accomplish the foregoing without a large paradigm shift in the image-review methodology already familiar to radiologists.

A number of exemplary scenarios for addressing the aforementioned issue(s) are now detailed along with an exemplary disadvantage(s) for each.

Scenario 1: Review every thin slice image of a high resolution scan; Disadvantages: There may be an impractical number of images to review; the radiologist may become fatigued should they try to review every image; and the signal-to-noise of the images may be increased. Additionally, using a rapid scrolling or flying through the images as an exclusive means of reviewing cases with a large number of images may lead to a decrease in diagnostic accuracy.

Scenario 2: Review thick slices on a traditional x-ray film and thin slices from a CT scan on a review station;

Disadvantage: This does not provide an easy means to relate and make transitions between thick and thin slices; and doctors may opt not use the review station after they have read the films.

Scenario 3: Multiple scans (e.g., one high resolution scan with a larger number of images/slices and one thick slice scan with fewer images);

Disadvantage: Inefficient work flow; and, the radiologist still does not know which of the high resolution images/slices to review.

The present invention contemplates a new and improved method and/or apparatus for reviewing tomographic scans with a large number of images which overcomes the above-referenced problems and others.

### BRIEF SUMMARY OF THE INVENTION

In accordance with one aspect of the present invention, a diagnostic medical imaging system is provided. It includes an imaging apparatus having an examination region in which a subject being examined is portioned. The imaging apparatus obtains, at first resolution, a plurality of first image slices of the subject. The first image slices are loaded into a storage device, and a data processor combines subsets of first image slices to generate a plurality of second image slices having a second resolution lower than the first resolution. The subsets each includes a number n of contiguous first image slices. A display having a plurality of view ports including a first view port which depicts one or more selected second image slices and a second view port which depicts one or more first image slices which are constituents of one of the second image slices depicted in the first view port.

In accordance with another aspect of the present invention, a diagnostic medical imaging system for examining a subject is provided. The diagnostic medical imaging system includes: acquisition means for obtaining a plurality of first image slices of the subject, the first image slices corresponding to a first thickness; combining means for generating a plurality of second image slices from combined subsets of first image slices, the subsets including a number n of contiguous first image slices, and the second image slices corresponding to a second thickness which is n times the first thickness; first display means for displaying one or more selected second image slices; and, second display means for displaying one or more of the first image slices included in the subset used to generate one the second image slices being displayed by the first displaying means.

In accordance with another aspect of the present invention, a method of diagnostic medical imaging includes: obtaining a plurality of first image slices of a subject, the first image slices corresponding to a first thickness; generating a plurality of second image slices from subsets of first image slices, the subsets including a number of contiguous first image slices, and the second image slices corresponding to a second thickness greater than the first thickness; designating regions of the subject for close review by a reviewer; sequentially displaying the second image slices for review by the reviewer; and, displaying the first image slices for review by the reviewer when the designated regions are reached.

One advantage of the present invention is that it presents a sequential image review format which is familiar to radiologists.

Another advantage of the present invention is that less images are presented for review compared to conventional review methods wherein all the high resolution images are reviewed.

Yet another advantage of the present invention is that higher resolution review may be urged and/or carried out in accordance with departmental policy or as desired by the radiologist when circumstances are appropriate.

Still further advantages and benefits of the present invention will become apparent to those of ordinary skill in the art upon reading and understanding the following detailed description of the preferred embodiments.

### BRIEF DESCRIPTION OF THE DRAWING(S)

The invention may take form in various components and arrangements of components, and in various steps and arrangements of steps. The drawings are only for purposes of illustrating preferred embodiments and are not to be construed as limiting the invention.

FIG. 1 is diagrammatic illustration of an exemplary diagnostic medical imaging system in accordance with aspects of the present invention.

FIG. 2 depicts a first view port showing an array of thick slices in accordance with aspects of the present invention.

FIG. 3 depicts, in accordance with aspects of the present invention, a second view port showing a montage of constituent thin slices for the thick slice selected in FIG. 2.

FIG. 4 depicts, in accordance with aspects of the present invention, a third view port showing a reference image with the locations of the thick and thin slices shown in FIGS. 3 and 4 indicated therein.

FIG. 5 is a flow chart illustrating an exemplary image acquisition and sequential review process in accordance with aspects of the present invention.

FIG. 6 diagrammatic illustration showing a small object detection and highlighting feature in accordance with aspect of the present invention.

### DETAILED DESCRIPTION OF THE PREFERRED EMBODIMENT(S)

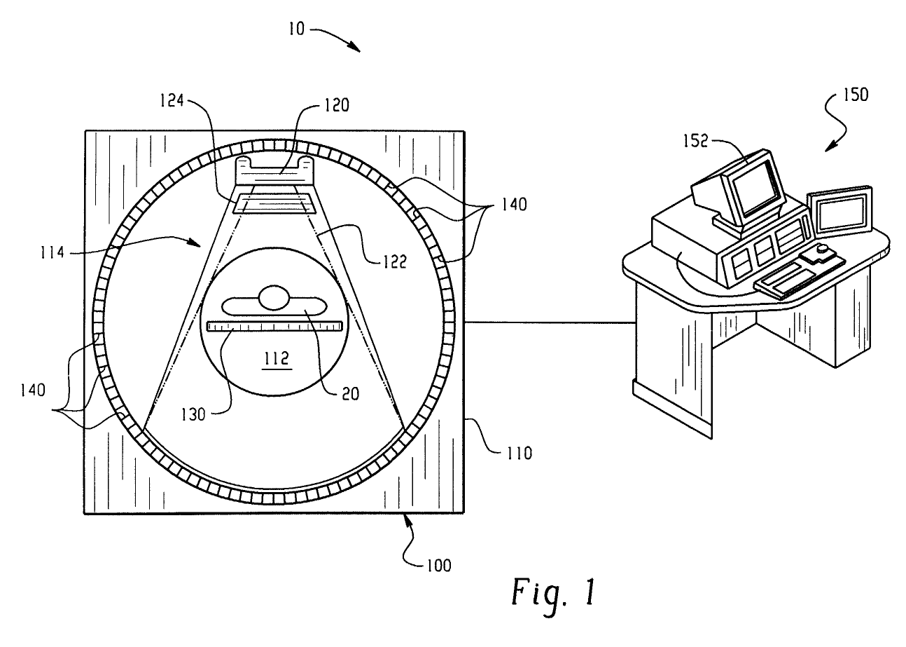

With reference to FIG. 1, a diagnostic medical imaging system 10 includes an imaging apparatus 100 capable of generating a plurality of diagnostic medical images of a subject 20. Preferably, the imaging apparatus 100 is a multi-slice or multi-detector CT scanner. Optionally, the imaging apparatus 100 is some other diagnostic medical imager, such as, an x-ray imaging device, a CT scanner, MRI scanner, fluoroscope, SPECT scanner, PET scanner, or the like, which is capable of generating multiple image slices.

In the illustrated embodiment, the diagnostic imaging apparatus 100 is a multi-slice CT scanner having a stationary gantry 110 which defines a central examination region 112. A rotating gantry 114 is mounted on the stationary gantry 110 for rotation about the examination region 112. A source of penetrating radiation 120, such as an x-ray tube, is arranged on the rotating gantry 114 for rotation therewith. The source of penetrating radiation produces a beam of radiation 122 that passes through the examination region 112 as the rotating gantry 114 rotates. A collimator and shutter assembly 124 forms the beam of radiation 122 and selectively gates the beam 122 on and off. Alternately, the radiation beam 122 is gated on and off electronically at the source 120. Using an appropriate reconstruction algorithm in conjunction with the data acquired from the CT scanner, images of the subject 20 therein are selectively reconstructed.

A subject support 130, such as an operating table, couch or the like, suspends or otherwise holds the subject 20 received thereon, such as a human or animal patient, at least partially within the examination region 112 such that the beam of radiation 122 cuts a cross-sectional slice(s) through the region of interest of the subject 20.

In the illustrated fourth generation CT scanner, a plurality of rings of radiation detectors 140 are mounted peripherally around the examination region 112 on the stationary gantry 110. Alternately, a third generation CT scanner is employed with a plurality of arcs of radiation detectors 140 mounted on the rotating gantry 114 on a side of the examination region 112 opposite the source 120 such that they span the arc defined by the beam of radiation 122. Regardless of the configuration, the radiation detectors 140 are arranged to receive the radiation emitted from the source 120 after it has traversed the examination region 112. The rings or arcs are disposed axially with respect to one another along a central longitudinal axis of the examination region 112, i.e., the axis of rotation or z-axis. Each ring or arc preferably collects data representing one slice of a multi-slice scan.

In a source fan geometry, an arc of detectors which span the radiation emanating from the source 120 are sampled concurrently at short time intervals as the source 120 rotates behind the examination region 112 to generate a source fan view. In a detector fan geometry, each detector is sampled a multiplicity of times as the source 120 rotates behind the examination region 112 to generate a detector fan view. The paths between the source 120 and each of the radiation detectors 140 are denoted as rays.

The radiation detectors 140 convert the detected radiation into electronic projection data. That is to say, each of the radiation detectors 140 produces an output signal which is proportional to an intensity of received radiation. Optionally, a reference detector may detect radiation which has not traversed the examination region 112. A difference between the magnitude of radiation received by the reference detector and each radiation detector 140 provides an indication of the amount of radiation attenuation along a corresponding ray of a sampled fan of radiation. In either case, each radiation detector 140 generates data elements which correspond to projections along each ray within the view. Each element of data in the data line is related to a line integral taken along its corresponding ray passing through the subject being reconstructed.

With each scan by the CT scanner, the data from the radiation detectors 140 in each ring or arc is collected and reconstructed into an image representation of the subject 20 in the usual manner. For example, a data processing unit incorporated in a workstation and/or control console 150 collects the data from the detectors 140 and reconstructs the image representations or image data therefrom using rebinning techniques, convolution/backprojection algorithms, and/or other appropriate reconstruction techniques. The control console 150 is optionally remotely located with respect to the imaging apparatus 100 (e.g., in a shielded room adjacent the scanning room containing the imaging apparatus 100) and typically it includes: one or more monitors 152 or other human viewable display devices; a computer and/or data processing hardware and/or software; one or more memories or other data storage devices; and, one or more standard input devices (e.g., keyboard, mouse, trackball, microphone for use with a voice recognition processor or software, etc.) for user interface with the system 10.

In a preferred embodiment, each of the images or image data, corresponding to the axial cross-section slice defined by each ring or arc of detectors 140, is loaded and/or stored in an image memory or other like data storage device which is part of the workstation 150. Herein, the individual slices or axial cross-section images originally generated and/or collected by the multi-slice CT scanner or other multi-slice imaging apparatus 100 will be referred to as thin slices. The thin slices preferably have a thickness along the z-axis of approximately 0.5 mm.

In a preferred embodiment, after all the thin slices are acquired for a given scan, a number n of contiguous thin slices are combined to create what is referred to herein as a thick slice. As opposed to generating the thick slices after all the thin slices ave been acquired, each next thick slice may be generated in pipe line fashion, i.e., each next thick slice is generate after its constituent thin slices are acquired without waiting for the remaining thin slices to be acquired. Optionally, the value of n is adjustable or selectable by the operator of the system 10 via the control console 150. The resolution or thickness of the thick slice is then n×the thin slice thickness. Preferably, n is approximately 4 and the thick slice thickness is approximately 2.0 mm. The thick slice is a uniformly weighted average of the constituent thin slices in a preferred embodiment. Alternately, other similar combination methods may be employed as desired. The pre-computed thick slices are preferably also loaded and/or stored in an image memory or other like data storage device which is part of the workstation 150. Alternately, to conserve memory, the thick slices are not pre-computed, rather they are generated from the thin slices on command or as otherwise desired.

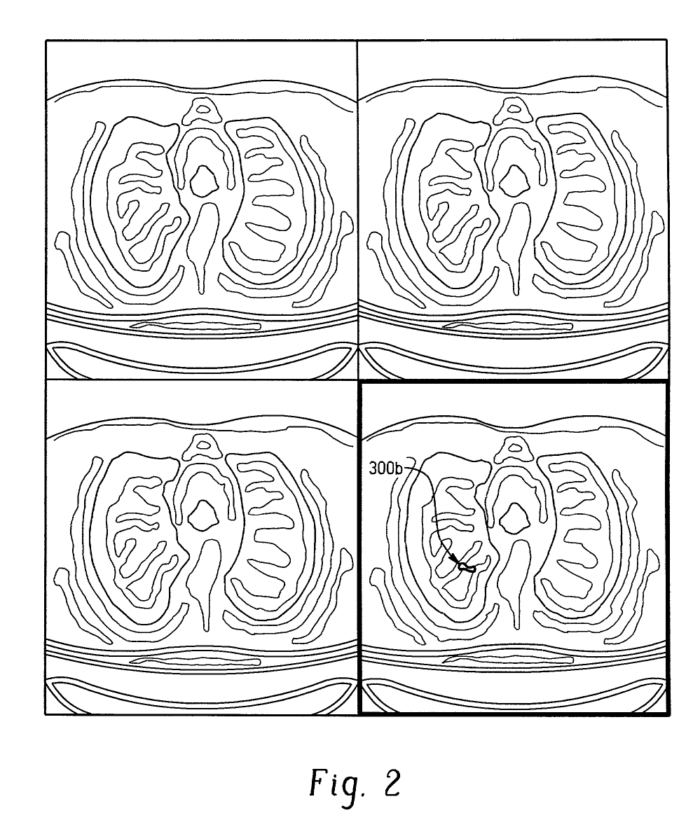

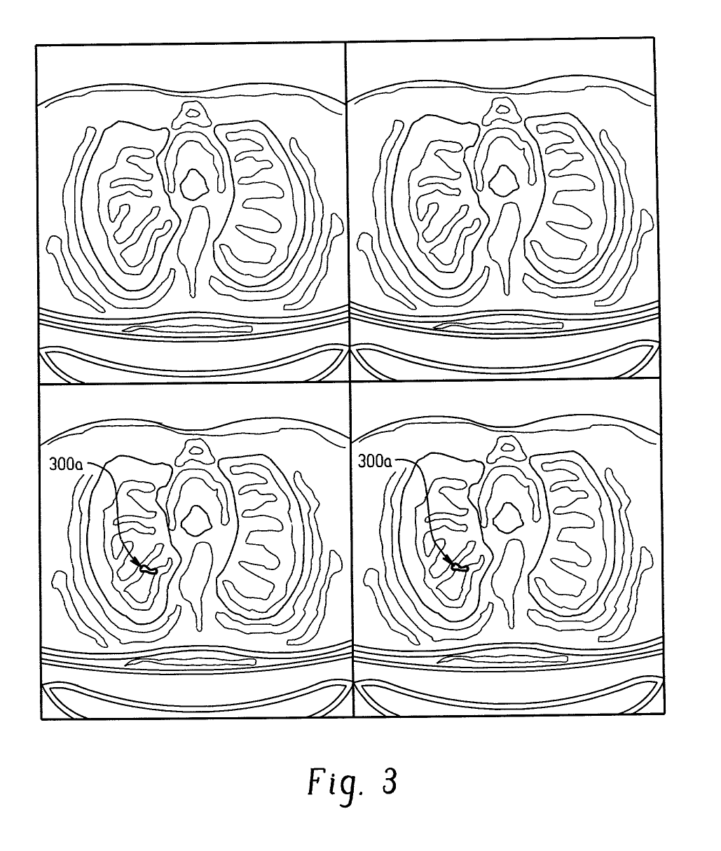

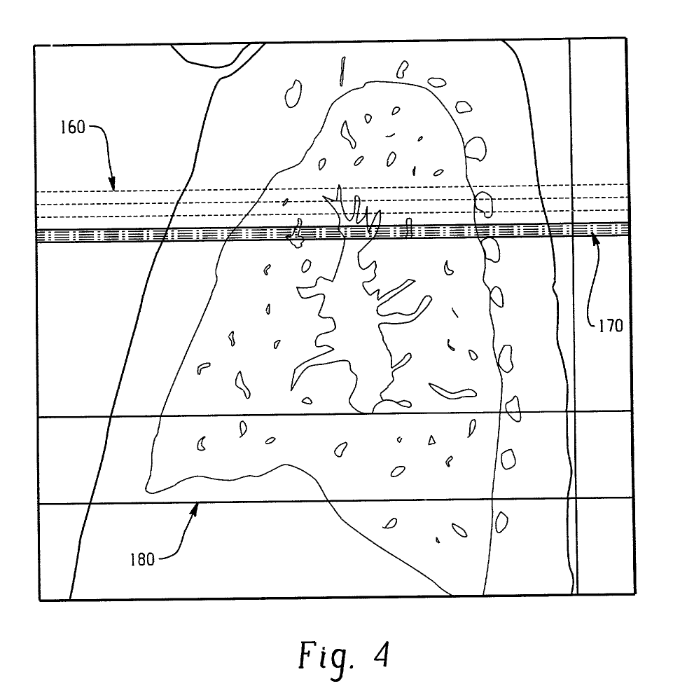

With reference to FIGS. 2 through 4, the thin and thick slices are selectively displayed on a human viewable display, such as a video monitor 152 or the like, which is also part of the console 150. At any given time, one or more view ports depicting various image representations of the subject 20 may be displayed on one or more of the monitors 152 or other like human viewable displays. Preferably, the view ports are provided with a graphical user interface (GUI) via which an operator and/or radiologist may carry out their review of and/or otherwise manipulate the acquired and/or generated images. Each view port may be on a separate dedicated monitor 152 or in a distinct window or other similarly defined region which shares a monitor 152. In accordance with one embodiment, there are preferably at least three view ports, namely: a first view port (as best seen in FIG. 3) which depicts the thick slices; a second view port (as best seen in FIG. 4) which depicts the constituent thin slices for the thick slice in the first view port; and a third view port (as best seen in FIG. 2) which depicts a multi-planar reformatted (MPR) view or image representation of the subject 20 viewed from a direction transverse to the viewing direction of the thick and thin slices. Preferably, e.g., where the thick and thin slices represent axial cross-section views, the MPR view is the corresponding coronal, sagittal or other like transverse view. Optionally, the first view port shows an array of (e.g., four (4)) contiguous thick slices, in which case, the second view port shows the constituent thin slices of the thick slice which is “selected” in the first view port. The thick slice may be selected by the operator or radiologist via the GUI or otherwise. As shown in FIG. 2, the selected thick slice is designated by a thickened or highlighted border.

In a preferred embodiment, the second view port shows a montage of the thin slices for the corresponding thick slice shown or selected in the first view port. Selection of a desired thin slice for closer inspection or review then optionally fills the second view port with a view of only the selected thin slice. Alternately, the thin slices are display in the second view port one at a time and are scrolled or otherwise paged through as desired. In another preferred embodiment, rather than showing the thin slices in a second view port, the thin slices are shown in the first view port when a thick slice is selected for closer inspection or review.

In any event, each view port preferably has graphical representations (e.g., points, cross-hairs, lines, etc.) of the contents of the other view ports for interpretation, reference and/or control. The third view port preferably has indicated therein the relative locations of the thick and/or thin slices in the image depicted in the third view port. For example, the thick slice(s) displayed in the first view port are indicated by a rectangular box(es) 160 located at its/their corresponding position(s) in the image of the third view port. Where multiple thick slices are shown in the first view port, the selected thick slice is preferably represented in or highlighted with a different color and/or line style. For example, non-selected thick slices may be indicated with a broken line rectangular box of a first color, while the selected thick slice is indicated with a solid line rectangular box of a second color. Similarly, the thin slices shown in the second view port are, e.g., shown by lines 170 superimposed at their corresponding locations in the image of the third view port. Similar to the thick slices, thin slices may also be highlighted or selected by the operator or radiologist via the GUI or otherwise. Again similar to the thick slices, the selected thin slice may be represented in or highlighted with a color and/or line style different from the non-selected thin slices. Additionally, the thickness of the thick slices is optionally determined by adjusting or setting the dimensions of the box 160 representing the same to encompass the desired number n of thin slices.

Preferably, the view ports are cross-referenced to one another such that the selection of a point (via, the GUI or otherwise) in the image of any one view automatically updates and/or reformats the complementary images of the other view ports to depict the corresponding point. Examples of view port cross-referencing are found in commonly owned U.S. Pat. Nos. 5,371,778 and 5,734,384 to Yanof, et al., both incorporated herein by reference in their entirety.

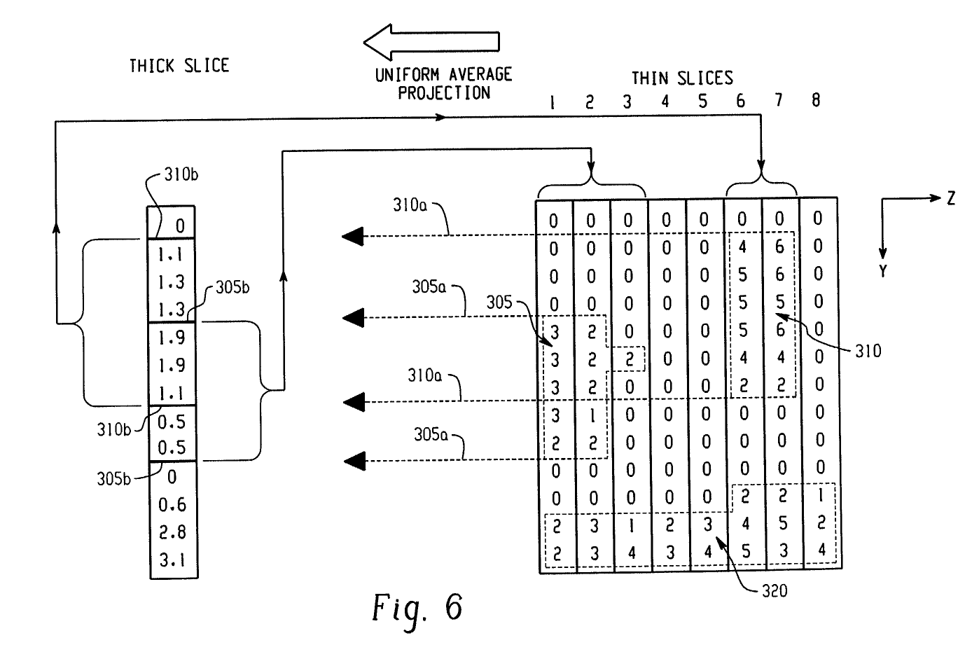

FIGS. 2 and 3, also indicate an exemplary small object via outlines 300a in the thin slices which are projected to the thick slice as outline 300b. This is described in more detail later herein with reference to FIG. 6.

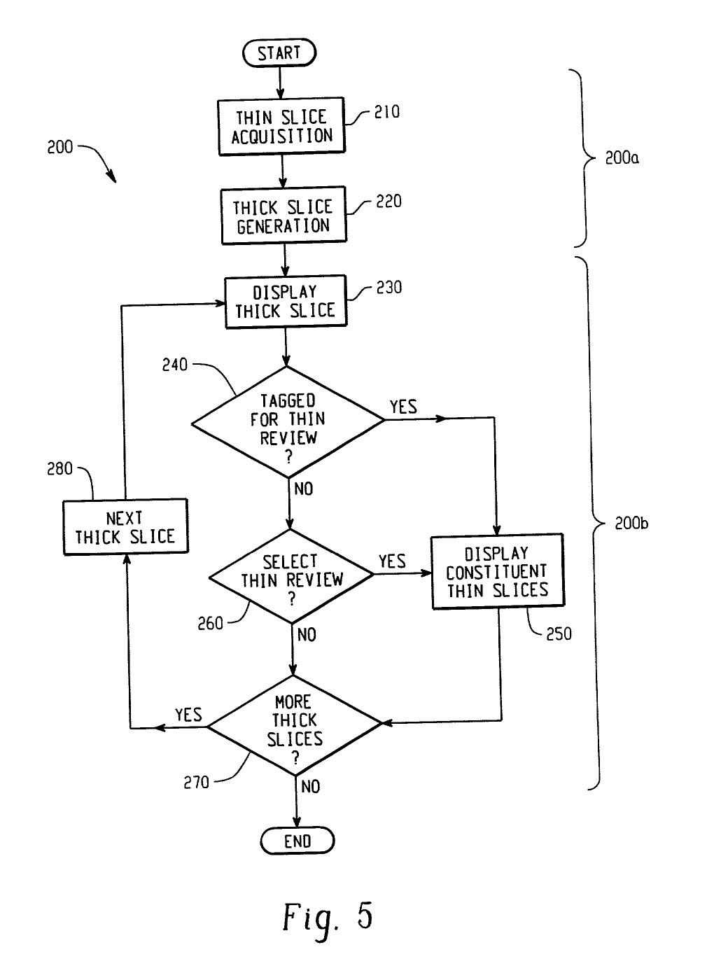

With reference to FIG. 5 and continuing reference to the preceding figures, an exemplary image acquisition and review procedure 200 employing the system 10 will now be described. An image acquisition and preparation phase 200a of the process 200 preferably includes steps 210 and 220. At step 210, the thin slices are acquired with the imaging apparatus 100, and at step 220 the thick slices are generated from the acquired thin slices. Optionally, in the image acquisition and preparation phase 200a of the process 200, the technologist or other operator carrying out the phase 200a, delineates, designates or tags a selected region or regions for thin slice review. The desired thin and/or thick slices are so designated or tagged. For example, this may be carried out by setting appropriate flags or labeling the appropriate data headers. Alternately, the data may be designated by specifying each thin slice review region as extending between two points or coordinates along the axial direction, in which case each thin and/or thick slice falling therebetween is marked as being in a thin slice review region. Preferably, the technologist or other operator acquiring the image data is prompted to designate the thin slice review regions, and he does so in accordance with a departmental policy or so that regions containing anatomy typically inspected at higher resolutions (e.g., the pancreas) are tagged for thin slice review. Optionally, the technologist may designate thin slice review regions in the third view port by highlighting or outlining the desired regions using the GUI or otherwise. See, e.g., box 180 in FIG. 4 designating an exemplary thin slice review region.

A sequential image review phase 200b of the process 200, is preferably conducted by the radiologist or other similar medical professional and preferably including steps 230 through 280. At step 230, the first thick slice or set of thick slices is displayed in the first view port for review. At decision step 240, it is determined if any of the displayed thick slices or their constituent thin slices have been tagged for thin slice review, i.e., if they fall within a thin slice review region. If the determination is yes or positive, the process 200 branches to step 250, otherwise if the determination is no or negative, the process continues on to decision step 260. At decision step 260, it is determined if the radiologist wants to voluntarily or otherwise desires to make a closer inspection or review of any of the displayed thick slices. The radiologist preferably so indicates by selecting the desired thick slice from the first view port. If the determination at decision step 260 is yes or positive, the process 200 branches to step 250, otherwise if the determination is no or negative, the process continues on to decision step 270.

At step 250, the constituent thin slices of the instant or selected thick slice are displayed in the second view port for review by the radiologist. Preferably, when step 250 is reached via a positive determination at decision step 240, the radiologist is alerted that the thin slices are in a designated thin slice review region, e.g., by a visual message displayed on one or more of the view ports and/or by another indication. Accordingly, the radiologist is urged and/or will scroll or page through or otherwise review each thin slice. Optionally, the radiologist may have the option of overriding selected thin slices review designations as he see fit.

At decision step 270, it is determined if any more thick slices remain for review. If the determination is yes or positive, the process continues with step 280, otherwise, if the determination is no or negative the process 200 ends. At step 280, the process 200 advances to the next thick slice or set of thick slices and then loops back to step 230 which then displays the current thick slice or set of thick slices. Preferably, the radiologist signals that he has completed his review of each thick or thin slice or set thereof by using the GUI to page down, scroll or otherwise advance to the next view.

Accordingly, via the process 200, a sequential review of the diagnostic images is achieved in a manner which is familiar to radiologists. Additionally, by view thick slices when high resolution is not desired, the time burden on the radiologist is reduced insomuch as fewer images are review. However, diagnostic accuracy is still maintained insomuch as the radiologist has the benefit of reviewing the higher resolution thin slices when selected regions are so indicated for such a review or the radiologist otherwise desired to do so. Of course, while the process 200 presents the radiologist with a sequential review of the images, the radiologist is also free to conduct a review the images randomly or as otherwise desired for more in depth inspection. Preferably, by employing the GUI or otherwise, the radiologist can selected desired points or regions or interest in any order for inspection, and in response to this selection, the view ports will automatically update and/or reformat the images depicted therein to show the selected point or region. In this manner then, the radiologist to free to go directly to a specific area of interest, e.g., for further review or to jump to a known area of concern.

To increase the diagnostic sensitivity of thick slices, the radiologist or other operator of the system 10 optionally invokes a small object detection and highlighting (SODH) feature of the system 10. SODH projects small objects from constituent thin slices into their corresponding thick slice. A small object for SODH purposes is any object having a dimension in the direction of slice thickness that is less than the thickness of the thick slice. Absent SODH, small objects may become obscured, lost or otherwise imperceivable when the thin slices are combined or project into the thick slice. That is to say, the relatively decreased resolution of the thick slices as compared to the thin slices is not desirable for the visualization of small objects.

An exemplary SODH feature, in accordance with a preferred embodiment, will now be described with reference to FIG. 6. FIG. 6 shows eight (8) thin slices being combined into a single thick slice using a uniform average projection. As shown, the slices are being viewed from a transverse direction, i.e., a sagittal or coronal direction. The numbers in each thin slice column are the CT numbers for the given image element or pixel. The numbers in the thick slice column are the averages of the CT numbers from the thin slices for the corresponding row. The two shaded areas 305 and 310 represent small objects. Preferably, the boundaries or outline 305a and 310a of each small object in the constituent thin slices is determined using known image edge detection techniques. The defined or determined outline of each small object is then projected onto the thick slice as corresponding outlines 305b and 310b. Preferably, each small object and its corresponding outline is color coded to distinguish one small object from the next. In this manner, small objects contained with the constituent thin slices of a thick slice are visualized in the thick slice as color coded outlines which define the boundaries of the small objects. Optionally, the outlines of the small objects are also similarly visualized in the reference image shown in the third view port. In a preferred embodiment, when the radiologist selects an outline in a view port using the GUI or otherwise, the constituent thin slices containing the associated small object are displayed in the second view port.

It is to be noted that, preferably, a large object (i.e., an object whose dimension span the thickness of the thick slice) is not subject to SODH. For example, the shaded area 320 representing the large object does not have its outline project onto the thick slice. To the extent that the resolution of the thick slice is enough to sufficiently visualize large objects, there is no added advantage to projecting a large object's outline from the constituent thin slices to the corresponding thick slice. That is to say, objects larger than the thick slice thickness already appear or are readily discernable in the thick slice.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

Having thus described the preferred embodiment(s), the invention is now claimed to be:

1. A diagnostic medical imaging system comprising:
an imaging apparatus having an examination region in which a subject being examined is portioned, said imaging apparatus obtaining a plurality of first image slices of the subject, said first image slices having a first resolution;
a storage device into which the first image slices are loaded;
a data processor which combines subsets of first image slices to generate a plurality of second image slices having a second resolution lower than the first resolution, said subsets each including a number n of contiguous first image slices; and,
a display having a plurality of view ports including a first view port which depicts one or more selected second image slices and a second view port which depicts one or more first image slices which are constituents of one of the second image slices depicted in the first view port.

2. The diagnostic medical imaging system according to claim 1, wherein the data processor combines the subsets using a uniform averaging projection.

3. The diagnostic medical imaging system according to claim 1, wherein the display includes a third view port which depicts a reference image which is viewed from a direction transverse to the first and second image slices.

4. The diagnostic medical imaging system according to claim 3, wherein the third view port superimposes over the reference image depicted therein graphical representations of the relative locations of the first and second images slices shown in the first and second view ports, respectively.

5. The diagnostic medical imaging system according to claim 1, further comprising:
a storage device into which the second image slices are loaded.

6. A diagnostic medical imaging system for examining a subject, said diagnostic medical imaging system comprising:
acquisition means for obtaining a plurality of first image slices of the subject, said first image slices corresponding to a first thickness;
combining means for generating a plurality of second image slices from combined subsets of first image slices, said subsets including a number n of contiguous first image slices, said second image slices corresponding to a second thickness which is n times the first thickness;
first display means for displaying one or more selected second image slices; and,
second display means for displaying one or more of the first image slices included in the subset used to generate one the second image slices being displayed by the first displaying means.

7. The diagnostic medical imaging system of claim 6, further comprising:
third display means for displaying a reference image which includes superimposed therein graphical representations of the relative locations of the second and first image slices displayed by the first and second display means, respectively.

8. The diagnostic medical imaging system of claim 7, further comprising:
means for updating the display of the first, second and third display means in response to a selection of a point in one of the same, such that each of the first, second and third display means displays the selected point.

9. The diagnostic medial imaging system of claim 7, wherein the reference image is selected from a view consisting of a coronal view, a sagittal view, and a multi-planar reformatted view.

10. The diagnostic medical imaging system of claim 6, further comprising:
means for detecting small objects contained in the subsets of first image slices, said small objects having dimensions in the direction of slice thickness less than the second thickness; and,
for projecting outlines of detected small objects onto the second image slices corresponding to the respective subsets.

11. The diagnostic medical imaging system of claim 10, wherein the outlines of detected small objects are color coded to distinguish them from one another.

12. The diagnostic medical imaging system of claim 6, further comprising:
means for storing the first and second image slices.

13. The diagnostic medical imaging system of claim 6, further comprising:
means for sequentially progressing through the plurality of second image slices such that each in turn is displayed on the first display means for review.

14. The diagnostic medical imaging system of claim 13, further comprising:
means for designating regions for close review such that during the sequential progression, when a designated region is reached, a reviewer is directed to the first image slices for review.

15. A method of diagnostic medical imaging, said method comprising:
(a) obtaining a plurality of first image slices of a subject, said first image slices corresponding to a first thickness;
(b) generating a plurality of second image slices from subsets of first image slices, said subsets including a number of contiguous first image slices, said second image slices corresponding to a second thickness greater than the first thickness;
(c) designating regions of the subject for close review by a reviewer;
(d) sequentially displaying the second image slices for review by the reviewer; and,
(e) displaying the first image slices for review by the reviewer when the designated regions are reached.

16. The method according to claim 15, wherein step (b) includes:
combining the subsets of first image slices via uniform averaging projection.

17. The method according to claim 15, further comprising
displaying a reference image of the subject; and,
superimposing in the reference image graphical representations of the relative locations of displayed first and second image slices.

18. The method according to claim 15, further comprising:
detecting small objects contained within the subsets of first image slices; and,
projecting outlines of the detected small objects into the thick slices corresponding to their respective subsets.
